(19) United States
(12) Patent Application Publication
(10) Pub. No.: US 2005/0226764 A1
(43) Pub. Date: Oct. 13, 2005

(54) Method and system for decontaminating a clean-room

(76) Inventors: Claude Moirandat, Basel (CH); Volker Sigwarth, Bad Sackingen (DE)

Correspondence Address: WELSH & KATZ, LTD, 120 S RIVERSIDE PLAZA, 22ND FLOOR, CHICAGO, IL, 60606, US

(21) Appl. No.: 10/519,888

(22) PCT Filed: Jun. 25, 2003
(86) PCT No.: PCT/CH03/00418

§ 371 Date: Dec. 29, 2004

(30) Foreign Application Priority Data
Jul. 2, 2002 (CH) 1156/02

(51) Int. Cl. A61L 2/20

(52) U.S. Cl. 422/30; 422/292; 422/306; 422/62

(57) ABSTRACT

A system for decontaminating a clean-room has an H₂O₂ supply device for supplying the clean-room with H₂O₂ and an H₂O₂ breakdown device for effecting a chemical breakdown of H₂O₂ without catalyst in the clean-room. The H₂O₂ breakdown device comprises a supply vessel filled with gaseous agent which can be introduced via a gas line into the clean-room where it reacts with the H₂O₂. A valve is placed in the gas line with which the amount of the gaseous agent introduced into the clean-room can be introduced under open-loop or closed-loop control. Owing to the fact that the excess H₂O₂, that is to say the H₂O₂ which has not reacted with other materials in the clean-room during the decontamination is broken down in the clean-room itself, it need not be flushed out completely from the clean-room first and broken down afterwards.

### FIELD OF THE INVENTION

The present invention relates to a method for decontaminating a clean-room, in which the clean-room is supplied with gaseous H₂O₂, and also relates to a system for decontaminating a clean-room, which system comprises an H₂O₂ supply device for supplying the clean-room with H₂O₂.

### BACKGROUND

In the context of this description and the patent claims, decontamination is also taken to mean sterilization and disinfection. Clean-room means all rooms which can be tightly sealed, for example isolators, locks, microbiological safety workbenches, sterilizers and transfer containers for the pharmaceutical industry, cosmetics, chemistry, food technology, electronics, nuclear industry, experimental animal husbandry, medicine, etc.

In food technology, hydrogen peroxide (H₂O₂) has already been used for many years in liquid form as a decontamination agent. Since, in high concentrations (>3%) it can act corrosively on various materials, it has not immediately found an opening in clean-room technology. Since the beginning of the 1980s, the microbiocidal properties of H₂O₂ at small concentrations have been intensively studied. It was found that H₂O₂ in vapor form, even at low concentration (100-5000 ppm), can destroy not only bacteria and spores thereof, but also fungi, yeasts and viruses. Since H₂O₂ does not act selectively, it is widely usable. In addition to formalin and peracetic acid, H₂O₂ has therefore been used in the past for rapid and safe decontamination of clean-rooms.

A system for decontaminating a clean-room which comprises an H₂O₂ supply device for supplying the clean-room with H₂O₂ is disclosed, for example, in CH-A-689 178. This system, in one variant, has an evaporator unit, an H₂O₂ supply vessel and a conveying device for conveying liquid H₂O₂ from the H₂O₂ supply vessel to the evaporator unit. The H₂O₂ supply vessel is disposed outside the clean-room and is connected via flexible tubing to the evaporator unit which is disposed within the clean-room. To supply the clean-room with H₂O₂, liquid H₂O₂ is conveyed from the H₂O₂ supply vessel to the evaporator unit and there evaporated, after which it is distributed in the clean-room. This is performed until the decontamination concentration is achieved. This is, in the case of H₂O₂, approximately 100-5000 ppm and is usually maintained for approximately from 10 to 120 minutes. After the decontamination, an exhaust air flap valve is opened and the H₂O₂-containing exhaust air is flushed out of the clean-room and passed via an exhaust air channel, in which case, to reduce the emissions, a catalyst can be present in the exhaust air channel, which catalyst decomposes the H₂O₂, for example into H₂O and O₂. Recirculation of the H₂O₂-supplied air over a catalyst is also known.

It is disadvantageous in this decontamination method that the excess H₂O₂, if it is broken down, is broken down using a catalyst. In order to achieve sufficiently rapid breakdown times, relatively large amounts of catalyst are required, which is very expensive. A further disadvantage is that the catalysts used need to be regenerated. Furthermore, any H₂O₂ breakdown does not proceed until outside the clean-room, that is to say the H₂O₂ must first be flushed out of the clean-room. Complete flushing of H₂O₂ out of the clean-room is relatively difficult, since it partially condenses in the clean-room and adheres to surfaces. In order that a desired residual concentration of usually from 5 to 0.05 ppm can be achieved, generally a flushing time of at least one hour is required, even if the clean-room is heated to evaporate the condensed H₂O₂.

U.S. Pat. No. 4,756,882 discloses a method for sterilizing an article in which the article is supplied with gaseous H₂O₂ in a closed chamber. The H₂O₂ which is still present after sterilization is decomposed into water, oxygen and hydrogen by generating a plasma. Here also, to generate the plasma, a relatively large amount of energy must be introduced into the closed chamber from the outside.

U.S. Pat. No. 5,820,841 discloses a similar method for sterilizing an article. Again the article is supplied with gaseous H₂O₂ in a closed chamber and the H₂O₂ still present after the sterilization is decomposed by generating a plasma. Here also, to generate the plasma a relatively large amount of energy must be introduced into the closed chamber from outside.

### SUMMARY

In view of the disadvantages of the previously known above described methods and systems for decontaminating a clean-room, the following object underlies the invention. A method and a system for decontaminating a clean-room are to be provided, which are to make possible, in the least costly manner possible, a decontamination using H₂O₂ and then to achieve as rapidly as possible the desired residual H₂O₂ concentration.

This object is achieved by the inventive method and the inventive system, as are defined in the independent patent claims 1 and 8. Preferred variants result from the dependent patent claims.

The essence of the invention is that in a method for decontaminating a clean-room, the clean-room is supplied with gaseous H₂O₂ and, at a later timepoint, H₂O₂ still present in the clean-room is chemically broken down without catalyst by supplying at least one gaseous agent which reacts with the H₂O₂.

Owing to the fact that the excess H₂O₂, that is to say the H₂O₂ which has not reacted with other materials in the clean-room during the decontamination is broken down in the clean-room itself, it need not be flushed out completely from the clean-room first. Furthermore, H₂O₂ which is condensed in the clean-room need not be evaporated first, as a result of which a heating of the clean-room can be dispensed with. The time for flushing out the exhaust air can thus be reduced to a few minutes. Therefore, cycle times of less than 60 minutes for decontaminating and flushing out are achievable, which corresponds to a considerable reduction compared with the prior art.

Owing to the gas form of the agent, this is distributed well in the clean-room and also comes into contact with the H₂O₂ which is condensed on surfaces, so that it rapidly reacts with the H₂O₂ and breaks this down.

Finally, owing to the fact that the H₂O₂ is chemically broken down without catalyst, no expensive catalysts are required for the breakdown of H₂O₂ in the clean-room or in the exhaust air.

Advantageously, H₂O₂ residues in a product situated in the clean-room are subsequently broken down on the product in a targeted manner. This is, for example, of importance when a lower H₂O₂ concentration is wanted for the product than is present in the clean-room after the H₂O₂ breakdown, and can be performed with the same agents.

Preferably, the at least one gaseous agent is metered in such a manner that after the chemical breakdown of the H₂O₂ at most 1 ppm of H₂O₂ still remains in the clean-room. Such a residual concentration is not a problem.

Preferably, the at least one gaseous agent comprises ammonia (NH₃). This reacts with the H₂O₂ as follows:
3 H₂O₂+2NH₃→N₂+6H₂O

The ammonia reduces the H₂O₂, with N₂ and water which is primarily in gaseous form being exclusively formed, that is to say harmless environmentally compatible reaction products. Since no precipitate is formed, these breakdown products can be flushed out of the clean-room without a problem into the exhaust air channel which need not conform to any special requirements with regard to chemical resistance. Furthermore, the exhaust air which can also comprise ammonia residues, can be discharged into the open without further treatment, since in addition to the breakdown products, the ammonia itself is also environmentally compatible.

Ammonia under usual ambient conditions, is a gas, it is easy to meter and is freely available on the market. The conventional quality (>99.7%) is sufficient for the inventive application. Furthermore, only small amounts of ammonia are required, that is to say about 0.5 l of NH₃ gas per g of pure H₂O₂. The amount of H₂O₂ and ammonia used obviously depends on the volume of the clean-room, and can therefore differ widely. The space and power requirements for storing and introducing the ammonia into the clean-room are small. Overall, therefore the use of ammonia is significantly cheaper than the use of catalysts, in particular in procurement, but also in use.

Furthermore, ammonia has the advantage that, like H₂O₂, it has a high affinity to water, and is readily soluble therein. Condensed H₂O₂ takes up NH₃ gas very well and is rapidly broken down.

A further advantage of ammonia is that it can be used very well even in large clean-rooms.

Furthermore, in the case of optimum use of ammonia, it is not necessary to flush out the clean-room, since the resultant atmosphere in the clean-room corresponds to the desired conditions.

Ammonia generally reacts with H₂O₂ very rapidly. Practical experiments have found that, at 25-35° C., the reaction time is about 1-2 minutes. Since any interfering residual products will be present in gas form, they can also be rapidly flushed out from the clean-room. The cycle time for decontamination of the clean-room, the breakdown of the H₂O₂, and a flushing of the clean-room can be reduced to less than 60 minutes.

An advantage of ammonia is that it is environmentally compatible and the MAC value (maximum workplace concentration) is 50 ppm, which is significantly higher in comparison with H₂O₂. Ammonia residues are therefore less of a problem than H₂O₂ residues. Furthermore, the odor of ammonia is characteristic and gives a warning. Ammonia gas is therefore also used, for example, to test for leaks of the isolator comprising the clean-room, and any gloves which are present. These tests can be carried out in the inventive method at the end of the cycle directly before flushing out the clean-room.

Control of the ammonia introduction is simple. It can be based on detecting an excess of ammonia or of H₂O₂ in the clean-room using chemical indicators or sensors.

Preferably, ammonia is introduced into the clean-room in excess, so that the breakdown reaction takes place rapidly and as completely as possible.

A disadvantage of ammonia is that it is flammable. However, the concentration which is required in the inventive method is low and the ammonia is largely immediately broken down by the H₂O₂. Only a possible ammonia excess is critical. This is therefore advantageously kept so low that the ignition limit of 15% is not reached. The metering is such that the ammonia excess is at most 500 ppm.

As an alternative to, or in combination with, ammonia, hydrazine (N₂H₄) can be used as gaseous agent. This reacts with the H₂O₂ as follows:
2H₂O₂+N₂H₄→N₂+4H₂O

The at least one gaseous agent can also comprise ozone (O₃) . This reacts with the H₂O₂ as follows:
H₂O₂+O₃→2O₂+H₂O

Ozone in the inventive method is not used to accelerate the sterilization, but to break down the H₂O₂.

The use of gaseous hydrazine or ozone to break down the H₂O₂ is associated with similar advantages as the use of ammonia.

In addition, the H₂O₂ still present can be photochemically broken down by UV radiation. This usually takes place as follows:

$$2 {H}_{2} {O}_{2} {⟶}^{UV} {O}_{2} + 2 {H}_{2} O$$

The UV light is preferably generated in the clean-room by a UV lamp disposed in the clean-room. It preferably has a wavelength of 254 nm.

The inventive system for decontaminating a clean-room comprises an H₂O₂ supply device for supplying the clean-room with H₂O₂ and an H₂O₂ breakdown device for effecting chemical breakdown of H₂O₂ without catalyst in the clean-room, which has means for introducing into the clean-room at least one gaseous agent, in particular ammonia, hydrazine or ozone. This system makes it possible to carry out the above mentioned inventive method which is associated with the advantages described.

In a preferred variant, the means for introducing at least one gaseous agent comprise a supply vessel charged with gaseous agent, for example a gas bottle, or a generator for producing gaseous agent, a gas line from the supply vessel or the generator to the clean-room, and a valve for regulating the amount of gaseous agent flowing through the gas line. The amount of the gaseous agent introduced into the clean-room can thus be regulated by means of the valve. In addition, gas cartridges can also be used which comprise the required amount of gaseous agent. A valve and a control device can then be omitted.

In an advantageous variant, the H₂O₂ breakdown device additionally has means for generating UV light in the clean-room. These means comprise, for example, a UV lamp which generates UV light within the clean-room. Such UV lamps are part of the prior art.

Advantageously, the inventive system has a sensor for measuring the concentration of the gaseous agent in the clean-room, the measured values of which serve to control the H₂O₂ breakdown device. If an excess of gaseous agent is measured which is not broken down by reaction with H₂O₂, the introduction of gaseous agent into the clean-room is usually stopped.

Instead of the quantitative sensor mentioned, a qualitative indicator, for example color indicator, is also conceivable. The breakdown process can also be controlled manually in this manner.

Alternatively or in addition, the inventive system has a sensor for measuring the H₂O₂ concentration in the clean-room, the measured values of which serve to control the H₂O₂ breakdown device. If the sensor measures an H₂O₂ concentration in the clean-room which is less than the sought-after residual concentration, for example 1 ppm, the H₂O₂ breakdown no longer needs to be advanced. This means that no additional gaseous agent needs to be introduced into the clean-room, or no additional UV light needs to be generated in the clean-room.

For open-loop control or closed-loop control of the H₂O₂ supply device, and of the H₂O₂ breakdown device, preferably separate open-loop control and closed-loop control devices are provided which makes possible subsequent installation of the H₂O₂ breakdown device into an existing system having H₂O₂ supply device.

The H₂O₂ breakdown device can either be constructed as a separate device which, independently of the H₂O₂ supply device, introduces gaseous agent into the clean-room, or generates it in this, or it and the H₂O₂ supply device can be integrated into a periphery of the clean-room. In new decontamination apparatuses, generally, the integration of the H₂O₂ breakdown device and the H₂O₂ supply device into the periphery of the clean-room is preferred, while it is simpler to retrofit existing decontamination apparatuses with a separate H₂O₂ breakdown device.

### BRIEF DESCRIPTION OF THE DRAWINGS

The inventive system for decontaminating a clean-room is described in more detail hereinafter with reference to the accompanying drawings on the basis of two example embodiments. In the drawings:

FIG. 1 diagrammatically shows a first example embodiment of the inventive system having a separate H₂O₂ breakdown device; and

FIG. 2 diagrammatically shows a second example embodiment of the inventive system having an H₂O₂ supply device and H₂O₂ breakdown device integrated into a periphery of the clean-room.

### DETAILED DESCRIPTION

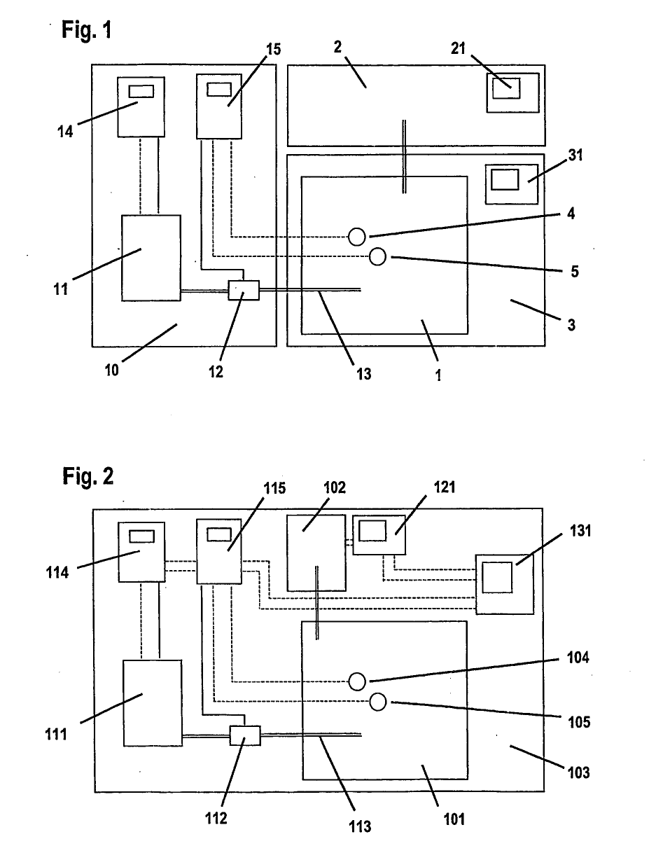

In the first example embodiment shown in FIG. 1 of an inventive system for decontaminating a clean-room 1, an H₂O₂ supply device 2 is disposed outside a periphery 3 of the clean-room 1. An open-loop control and close-loop control device 31 controls the conditions in clean-room 1, in particular the pressure relationships and the air conditions. The H₂O₂ supply device 2 comprises, for example, as described in CH-A-689 178 at least one liquid-H₂O₂-filled H₂O₂ supply vessel, at least one evaporator unit in the form of a heating plate for vaporizing the H₂O₂ and at least one H₂O₂ line between the at least one H₂O₂ supply vessel and the at least one heating plate. The at least one heating plate is disposed in the clean-room 1, so that the H₂O₂ which is fed from the at least one H₂O₂ supply vessel via the at least one H₂O₂ line is vaporized directly in the clean-room 1 on the at least one heating plate. The supply of H₂O₂ to the clean-room 1 is controlled by an open-loop control and closed-loop control device 21 which preferably comprises a stored-programmable control. Usually, sufficient H₂O₂ is vaporized in the clean-room 1 so that in clean-room 1 an H₂O₂ concentration of approximately 100-5000 ppm is present for approximately from 10 to 120 minutes.

After the decontamination with H₂O₂, the H₂O₂ still present in the clean-room 1, that is to say the H₂O₂ which has not reacted and has not been consumed, is broken down using a gaseous agent which is introduced into the clean-room 1 via a gas line 13. As gaseous agent, use is preferably made of either ammonia, hydrazine or ozone.

For this purpose, the system has a separately constructed H₂O₂ breakdown device 10 which comprises a supply vessel 11 in which the gaseous agent is stored. The stock of gaseous agent in the supply vessel 11 is monitored by a control unit 14. The gaseous agent stored in the supply vessel 11 passes into the clean-room 1 via the gas line 13, in which case one or more nozzles can be provided at the clean-room-side end of the gas line 13, which nozzles distribute the gaseous agent in the clean-room 1. In the gas line 13, there is disposed a valve 12 with which the amount of the gaseous agent introduced into the clean-room 1 can be introduced under open-loop or closed-loop control. The valve 12 is controlled via an open-loop control and closed-loop control device 15 which is connected to a sensor 4 for measuring the concentration of the gaseous agent and to a sensor 5 for measuring the H₂O₂ concentration. The sensors 4 and 5 are disposed in the clean-room 1 and measure the concentration of the gaseous agent and the H₂O₂ concentration in the clean-room 1.

Depending on the values measured by the sensors 4 and 5, more or less gaseous agent is fed to the clean-room 1. Generally, a small excess of gaseous agent is introduced into the clean-room 1, so that the H₂O₂ is broken down rapidly and as completely as possible.

After the breakdown of the H₂O₂, in the clean-room 1 the air exchange is ensured again, in which case for this purpose in a known manner a feed air channel, a feed air flap valve, an exhaust air flap valve and an exhaust air channel can be provided. The system can in addition have further elements which are known from systems for decontaminating a clean-room of the prior art.

In the second embodiment example shown in FIG. 2 of an inventive system for decontaminating a clean-room 101, the H₂O₂ breakdown device and the H₂O₂ supply device 102 are integrated into the periphery 103 of the clean-room 101. The H₂O₂ breakdown device comprises, instead of a supply vessel for gaseous agent, a gas generator 111 which generates the gaseous agent directly. The generator 111 is controlled by an open-loop control unit 114. The gaseous agent generated is fed via a gas line 113 to the clean-room 101, the amount of agent fed being introduced under open-loop or closed-loop control via a valve 112 disposed in the gas line 113. The valve 112 is controlled by an open-loop and closed-loop control device 115 which is connected to a sensor 104 for measuring the concentration of the gaseous agent and to a sensor 105 for measuring the H₂O₂ concentration. The sensors 104 and 105 are disposed in the clean-room 101 and measure the concentration of the gaseous agent and the H₂O₂ concentration in the clean-room 101.

The open-loop control and closed-loop control device 115 is also connected to the control unit 114 and via this ensures that gaseous agent is generated or not in correspondence with the measured values of the sensors 104 and 105.

As in the first example embodiment, the clean-room 101 is supplied with H₂O₂ under open-loop control and closed-loop control by an open-loop control and closed-loop control device 121 which preferably comprises a stored-programmable control. By means of an open-loop control and closed-loop control device 131, the conditions in the clean-room 101 are subjected to open-loop control and closed-loop control, in particular the pressure relationships and the air conditions. The open-loop control and closed-loop control device 121 is here connected via the open-loop control and closed-loop control device 131 to the open-loop control and closed-loop control device 115, so that the measured values of the sensors 104 and 105 can also be used for the open-loop control of the H₂O₂ supply.

Further, that said with reference to the first example embodiment applies.

Further structural variations can be implemented to the above described systems for decontaminating a clean-room. It may be further explicitly stated here that the H₂O₂ supply device can also be constructed differently than described. For example, H₂O₂ which is already gaseous could be introduced from outside into the clean-room 1 or 101. In principle, all H₂O₂ supply devices of the prior art are conceivable.

Specific embodiments of a method and system for decontaminating a clean room according to the present invention have been described for the purpose of illustrating the manner in which the invention may be made and used. It should be understood that implementation of other variations and modifications of the invention and its various aspects will be apparent to those skilled in the art, and that the invention is not limited by the specific embodiments described. It is therefore contemplated to cover by the present invention any and all modifications, variations, or equivalents that fall within the true spirit and scope of the basic underlying principles disclosed and claimed herein.

1. A method for decontaminating a clean-room, in which the clean-room is supplied with gaseous H₂O₂ and H₂O₂ still present in the clean-room is chemically broken down without catalyst at a later timepoint by supplying at least one gaseous agent which reacts with the H₂O₂.

2. The method as claimed in claim 1, in that wherein H₂O₂ residues in a product situated in the clean-room are subsequently broken down on the product in a targeted manner.

3. The method as claimed in claim 1, wherein the at least one gaseous agent is metered in such a manner that after the chemical breakdown of the H₂O₂ at most 1 ppm of H₂O₂ still remains in the clean-room.

4. The method as claimed in claim 1, wherein the at least one gaseous agent comprises ammonia.

5. The method as claimed in claim 4, that wherein the ammonia is metered as a function of the H₂O₂ in such a manner that the excess of ammonia is at most 500 ppm.

6. The method as claimed in claim 1, wherein the at least one gaseous agent comprises hydrazine.

7. The method as claimed in claim 1, wherein the at least one gaseous agent comprises ozone.

8. A system for decontaminating a clean-room having an H₂O₂ supply device for supplying the clean-room with H₂O₂, comprising an H₂O₂ breakdown device for effecting a chemical breakdown of H₂O₂ without catalyst in the clean-room, which further comprises means for introducing at least one gaseous agent into the clean-room.

9. The system as claimed in claim 8, wherein the means for introducing at least one gaseous agent is constructed to introduce ammonia, hydrazine or ozone into the clean-room.

10. The system as claimed in claim 8, wherein the means for introducing at least one gaseous agent into the clean-room has a supply vessel filled with gaseous agent, or a generator for generating gaseous agent, a gas line from the supply vessel or generator to the clean-room and a valve for regulating the amount of the gaseous agent flowing through the gas line.

11. The system as claimed in claim 8, further including a sensor for measuring the concentration of the gaseous agent in the clean-room, the measured values of which serve to control the H₂O₂ breakdown device.

12. The system as claimed in claim 8, further including a sensor for measuring the H₂O₂ concentration in the clean-room, the measured values of which serve to control the H₂O₂ breakdown device.

13. The system as claimed in claim 8, wherein the H₂O₂ breakdown device has means for generating UV light in the clean-room.

14. The system as claimed in claim 8, wherein the H₂O₂ breakdown device and the H₂O₂ supply device are integrated into a periphery of the clean-room.

15. (canceled)
